(19) United States
(12) Patent Application Publication
(10) Pub. No.: US 2005/0131273 A1
(43) Pub. Date: Jun. 16, 2005

(54) Relaxation system, relaxation method and relaxation program

(76) Inventors: Masakazu Asano, Osaka (JP); Hirokazu Genno, Osaka (JP)

Correspondence Address: McDERMOTT WILL & EMERY LLP, 600 13th Street, N.W., Washington, DC, 20005-3096, US

(21) Appl. No.: 10/963,752

(22) Filed: Oct. 14, 2004

(30) Foreign Application Priority Data
Oct. 16, 2003 (JP) 2003-356569

(51) Int. Cl. A61M 21/00; A61H 1/00

(52) U.S. Cl. 600/27; 601/49

(57) ABSTRACT

A relaxation system includes a main body and a remote controller. In the relaxation system, a user grasps the remote controller for manipulation, whereby information on the health condition of the user is acquired. The information on the health condition means, for example, voice data based on the voice of the user, physiological data representing the skin temperature of the user, pulse rate, GRS, and the like, and question and answer data involving the identification number of the user, age, height, weight, and mental condition. The main body estimates physical and mental conditions of the user based on the acquired information on the health condition for controlling the operation of kneading balls.

### BACKGROUND OF THE INVENTION

1. Field of the Invention

The present invention relates to a relaxation system, relaxation method, and relaxation program which relieve the stress of a human body.

2. Description of the Background Art

A variety of massage machines and relaxation apparatus have conventionally been developed in order to relieve the stress of a human body, and provide recovery from fatigue.

For example, a relaxation apparatus has been developed in which the speed of stimulating a human body with kneading balls is controlled with the respiration cycle of the user being measured, so that the stimulation speed is gradually slowed down starting from the usual speed, thereby achieving a relaxation effect (refer to JP 7-204238 A, for example). A massage machine has also been developed in which the kneading patterns are varied according to the stiffness or degree of a relaxed state of a human body (refer to JP 2002-233558, for example).

The physical and mental conditions of the user, however, vary from time to time. With the conventional massage machine and relaxation apparatus, it is difficult to relive the stress sufficiently, effectively, according to the physical and mental conditions of the user.

### SUMMARY OF THE INVENTION

It is an object of the present invention to provide a relaxation system, relaxation method, and relaxation program which allow a user to relieve the stress of his body sufficiently, effectively, according to the physical and mental conditions of the user.

A relaxation system according to one aspect of the present invention comprises: an acquisition device that acquires information on the health condition of a user; an estimator that estimates a physical condition and a mental condition based on the information acquired by the acquisition device; a stress relieving apparatus that relives the stress of the user; and a controller that controls the stress relieving apparatus based on estimation results by the estimator.

In the relaxation system, the stress relieving apparatus is controlled based on the estimation results on physical and mental conditions, so that the stress of a human body can be sufficiently, effectively relieved according to his physical and mental conditions.

The relaxation system may further comprise a correlation storage that stores in advance a first correlation between the information acquired by the acquisition device and the physical condition and a second correlation between the information acquired by the acquisition device and the mental condition, the estimator extracting the physical and mental conditions based on the first and second correlations stored in the correlation storage.

In this case, the estimator extracts the physical and mental conditions of the user with reference to the first and second correlations stored in the correlation storage, based on the information acquired by the acquisition device. This facilitates the estimation of physical and mental conditions of the user based on the information acquired by the acquisition device.

The acquisition device may include a physiological data acquisition device that acquires information on the physiological data of the user, the information on the health condition including the information acquired by the physiological data acquisition device.

In this case, the estimation of physical and mental conditions of the user can be done in more detail and more accurately, based on the information on the physiological data of the user.

The acquisition device may include a voice acquisition device that acquires information on the voice of the user, the information on the health condition including the information acquired by the voice acquisition device.

In this case, the estimation of physical and mental conditions of the user can be done in more detail and more accurately, based on the information on the voice of the user.

The acquisition device may include a questioning device that questions the user while inputting an answer to the question, the information on the health condition including the answer input by the questioning device.

In this case, since the acquisition device acquires the answer from the questioning device, the estimation of physical and mental conditions of the user can be done based on the subjective physical and mental conditions of the user. This results in a more detailed and accurate estimation of physical and mental conditions of the user.

The relaxation system may further comprise a storage that stores in advance a plurality of stress relieving techniques, the controller selecting any of the plurality of stress relieving techniques stored in the storage based on the physical and mental conditions estimated by the estimator, to control the stress relieving apparatus according to the selected stress relieving technique.

In this case, the controller selects a suitable stress reliving technique for the user among the plurality of stress relieving techniques based on the estimated physical and mental conditions, thereby controlling the stress relieving apparatus based on the selected stress reliving technique, allowing the user to relieve the stress of his body sufficiently, effectively, based on his physical and mental conditions.

The correlation storage may additionally store a third correlation between physical and mental conditions and a plurality of stress relieving techniques for relieving the stress of the user.

In this case, since the third correlation is stored in the correlation storage, the stress relieving technique corresponding to the physical and mental conditions of the user can be readily selected.

The controller may select any of the plurality of stress relieving techniques with reference to the third correlation stored in the correlation storage based on the physical and mental conditions estimated by the estimator, to control the stress relieving apparatus according to the selected stress relieving technique.

In this case, the controller selects one stress relieving technique with reference to the third correlation based on the estimated physical and mental conditions, thereby controlling the stress relieving apparatus according to the selected stress relieving technique, allowing the user to relieve the stress of his body sufficiently, effectively based on his physical and mental conditions.

The acquisition device may include a questioning device that questions the user while inputting an answer to the question, the information on the health condition may include the answer input by the questioning device, and the controller may modify the third correlation based on the answer input by the questioning device, to select any of the stress relieving techniques with reference to the modified third correlation based on the physical and mental conditions estimated by the estimator.

In this case, since the acquisition device acquires the answer from the questioning device as the information on the health condition, the estimation of physical and mental conditions of the user can be done based on the subjective physical and mental conditions of the user. This results in a more detailed and accurate estimation of physical and mental conditions of the user.

Further, the controller modifies the third correlation based on the answer acquired by the acquisition device to select one stress relieving technique based on the modified third correlation, thereby controlling the stress relieving apparatus according to the selected stress relieving technique. This allows the user to relieve the stress of his body sufficiently, effectively, based on his physical and mental conditions.

The acquisition device may include a questioning device that questions the user while inputting an answer to the question, the information on the health condition may include the answer input by the questioning device, the correlation storage may additionally store a plurality of third correlations between the physical and mental conditions and a plurality of stress relieving techniques for relieving the stress of the user, and the controller may select any of the plurality of third correlations based on the answer input by the questioning device, to select any of the plurality of stress relieving techniques with reference to the third correlation selected based on the physical and mental conditions estimated by the estimator, for controlling the stress relieving apparatus according to the selected stress relieving technique.

In this case, since the acquisition device acquires the answer from the questioning device as the information on the health condition, the estimation of physical and mental conditions of the user can be done based on the subjective physical and mental conditions of the user. This results in a more detailed and accurate estimation of physical and mental conditions of the user.

Moreover, the controller selects one of the plurality of third correlations stored in the correlation storage based on the answer acquired by the acquisition device, to select a stress relieving technique based on the selected third correlation for controlling the stress relieving apparatus according to the selected stress relieving technique. This allows the user to relieve the stress of his body sufficiently, effectively, based on his physical and mental conditions.

The relaxation system may further comprise an input device with which the user can select any of the plurality of third correlations, the controller controlling the stress relieving apparatus according to the stress relieving technique based on the third correlation selected by the user manipulating the input device.

In this case, since the user can select a third correlation, the user can relieve the stress of his body sufficiently, effectively to his own taste.

The stress relieving apparatus may include at least one of a respiration guidance device that controls the respiration of the user, a chaotic fluctuation vibrator that gives vibration based on chaotic fluctuation to the user, an illumination controller that controls the condition of illumination, and a music provider that provides the user with music.

In this case, at least one of the respiration guidance device, chaotic fluctuation vibrator, illumination controller, and music provider allows the stress of a human body to be relieved sufficiently, effectively, according to his estimated physical and mental conditions.

The stress relieving apparatus may be a massager. In this case, the user can receive a massage by the massager according to his physical and mental conditions. As a result, the stress of his body is sufficiently, effectively relieved.

A relaxation method according to another aspect of the present invention comprises the steps of: acquiring information on the health condition of a user; estimating a physical condition and a mental condition based on the acquired information; and relieving the stress of the user based on estimation results on the physical and mental conditions.

In the relaxation method, the stress relieving apparatus is controlled based on the estimation results on physical and mental conditions, which allows the user to relieve the stress of his body sufficiently, effectively according to his physical and mental conditions.

A relaxation program according to still another aspect of the present invention is a computer-executable program which allows the computer to execute the processes of: acquiring information on the health condition of a user; estimating a physical condition and a mental condition based on the acquired information; and relieving the stress of the user based on estimation results on the physical and mental conditions.

In the relaxation program, the stress relieving apparatus is controlled based on the estimation results on physical and mental conditions, which allows the user to relieve the stress of his body sufficiently, effectively according to his physical and mental conditions.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is an external perspective view showing an example of a relaxation system according to an embodiment of the present invention;

FIG. 2 is an external view of the remote controller of FIG. 1;

FIG. 3 is a block diagram showing the configuration of the relaxation system according to the embodiment of the present invention;

FIG. 4 is a diagram showing an example of a variety of tables and databases stored in the memory of FIG. 3;

FIG. 5 shows examples of the physical condition table and the mental condition table of FIG. 4;

FIG. 6 shows an example of the question table of FIG. 4;

FIG. 7 shows an example of the relaxation technique table of FIG. 4;

FIG. 8 is a table for use in illustrating a specific example of a method of determining a modification level;

FIG. 9 shows examples of a plurality of relaxation technique tables stored in the memory of FIG. 4;

FIG. 10 shows an example of a modification table for determining one relaxation technique table among the plurality of relaxation technique tables;

FIG. 11 is a flowchart showing an example of a relaxation program according to the embodiment of the present invention;

FIG. 12 is a flowchart showing an example of the relaxation program according to the embodiment of the present invention;

FIG. 13 is a schematic diagram showing an example of a display form on the display of FIG. 2 during the questioning to the user;

FIG. 14 is a diagram showing examples of chaotic fluctuation and on/off of the leg vibrator based on the chaotic fluctuation;

FIG. 15 is a block diagram showing an example of a relaxation system with a personal computer;

FIG. 16 is a diagram showing an example of a main screen provided on the display of FIG. 15; and

FIG. 17 is a diagram showing an example of a screen displayed when the relaxation system of FIG. 15 is performing a respiration guidance to the user.

### DESCRIPTION OF THE PREFERRED EMBODIMENTS

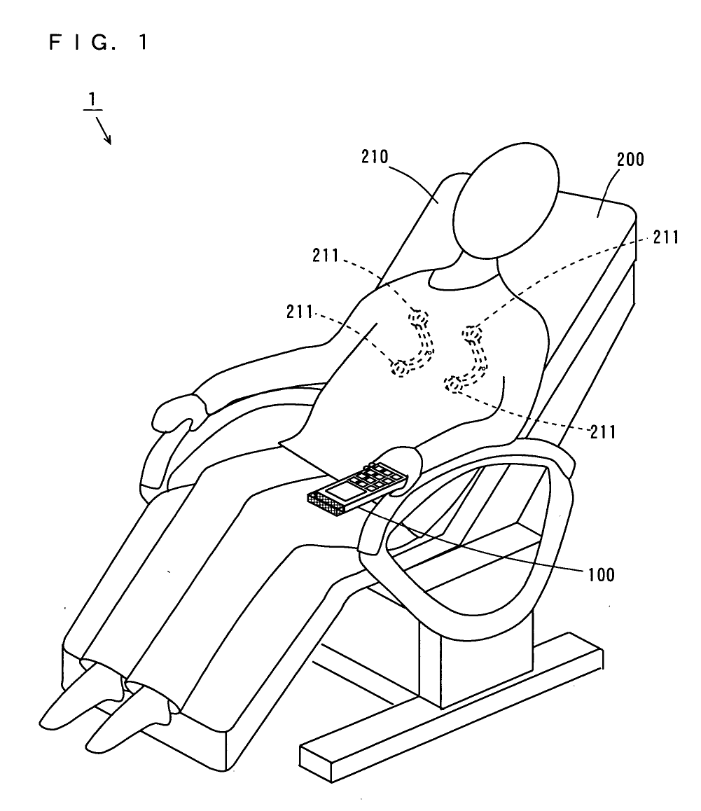

A relaxation system according to an embodiment of the present invention will, hereinafter, be described with reference to FIGS. 1 to 17. In the embodiment, description is given of a relaxation system using a massage chair as an example of the relaxation system.

FIG. 1 is an external perspective view showing an example of the relaxation system according to the embodiment of the present invention.

As shown in FIG. 1, the relaxation system 1 according to the embodiment comprises a main body 200 composed of a massage chair and a remote controller 100.

In the relaxation system 1 of FIG. 1, a user grasps the remote controller 100 for manipulation, whereby information on the health condition of the user is acquired. The information on the health condition means, for example, voice data obtained by analyzing the voice of the user; physiological data that can be objectively measured, such as the blood pressure level, blood glucose level, weight, waist value, or cholesterol value of the user; and question and answer data representing the user's answers to questions.

In the embodiment, the physiological data represents measurements of the skin temperature, pulse rate, and GRS (Galvanic Skin Response) of the user, whereas the Q&A data represents the user's answers to the questions on his personal information as well as physical and mental questions, that is, answers to such questions as the identification number (personal number), age, height, weight, and feeling of the user.

The voice data may be obtained, for example, by inputting the voice of the user to a voice collection microphone to be described later provided in the remote controller 100, and analyzing the voice.

The physiological data may be obtained, for example, when each of sensors to be described later provided in the remote controller 100 is brought into contact with the user.

The Q&A data may be obtained, for example, by displaying questions on a display to be described later provided in the remote controller 100, and requesting the user to answer the questions by manipulating an input unit described later in response to each question.

The main body 200 estimates physical and mental conditions of the user, based on one or more of the voice data, physiological data, and Q&A data acquired by the remote controller 100.

In the embodiment, the main body 200 estimates the current physical and mental conditions of the user, based on the voice data representing the tone of his spoken voice (intonation), inflection, jitter (degree of hoarseness), and the like obtained through the results of voice analysis, and the physiological data including the skin temperature, pulse rate, GRS, and the like obtained with the remote controller 100.

In the embodiment, a plurality of kneading balls 211 are incorporated into a backrest 210 of the main body 200 of FIG. 1. In addition, relaxation techniques according to a plurality of levels are set in advance in the main body 200 for controlling the operation of the kneading balls 211.

The main body 200 selects a preset relaxation technique level based on the physical and mental conditions estimated in the foregoing manner. Then, the main body 200 modifies the relaxation technique level selected based on the Q&A data obtained with the remote controller 100.

After that, the main body 200 controls the operation of the kneading balls 211 based on the selected relaxation technique level. This causes stimulation on the back of the user, thereby relieving the stress of his body sufficiently, effectively.

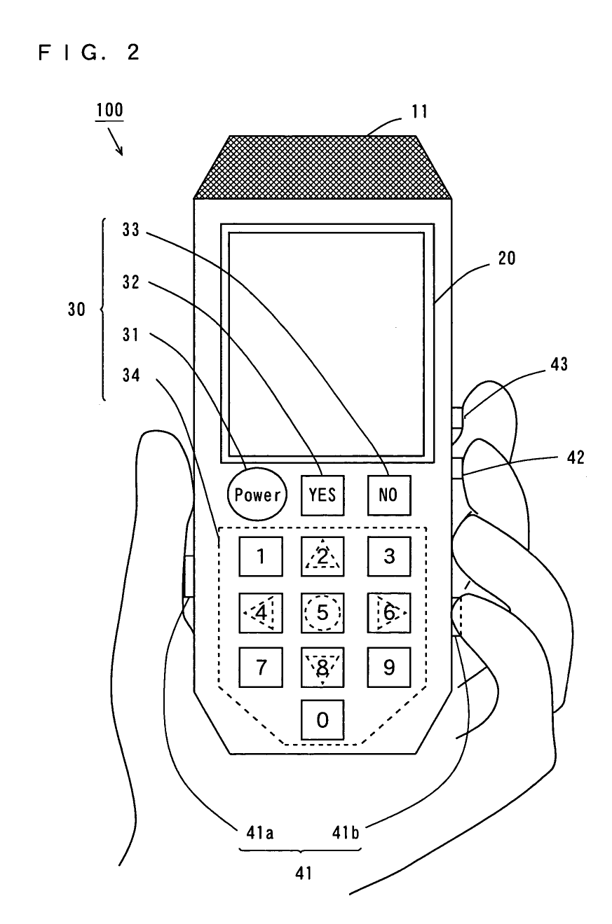

FIG. 2 is an external view of the remote controller 100 of FIG. 1. As shown in FIG. 2, the remote controller 100 according to the embodiment comprises a voice collection microphone 11, a display 20, an input unit 30, a GSR sensor 41, a pulse sensor 42, and a skin temperature sensor 43.

The input unit 30 as used herein includes a power supply button 31, a Yes button 32, a No button 33, and a plurality of numeric buttons 34. The GSR sensor 41 is composed of two electrodes, 41a, 41b.

In use, the user grasps the remote controller 100 of FIG. 1 as shown in FIG. 2. This allows the skin temperature sensor 43 to come into contact with the index finger, the pulse sensor 42 in contact with the ring finger, the one electrode 41a of the GSR sensor 41 in contact with the ring finger or little finger, and the other electrode 41b in contact with the thumb- or palm of the hand.

In FIG. 2, each of the variety of sensors on the remote controller 100 is arranged in a given position, assuming that the remote controller 100 is grasped by the left hand of the user; note, however, that the arrangement may be made such that it is grasped by the right hand.

The display 20 shows the instruction information for the user, physiological data, voice data, Q&A data, and the like.

When the display 20 provides an instruction for the user to speak a given word or sentence, the user speaks the given word or sentence toward the voice collection microphone 11. This causes the user voice to be input to the voice collection microphone 11 for voice analysis.

The input unit 30 will be described. The power supply button 31 is pressed when the user starts using the relaxation system 1. This switches on the relaxation system 1 (the remote controller 100 and main body 200).

The user presses the Yes button 32 and No button 33 in response to the contents provided on the display 20. The manipulation of the Yes button 32 and No button 33 will later be described.

The numeric buttons 34 as shown in FIG. 2 are provided with the numbers of “0” to “9”, respectively. The user presses any of the numeric buttons in response to the contents provided on the display 20.

Among the plurality of numeric buttons 34, the buttons assigned “2”, “4”, “6”, and “8” function as a cursor key, while the button assigned “5” functions as an Enter key. When the display 20 provides an item selection screen, the user presses any of the numeric buttons 34 assigned “2”, “4”, “5”, “6”, and “8”.

Each of the variety of sensors will be described. The skin temperature sensor 43 detects the skin temperature of the index finger in contact therewith. The pulse sensor 42 detects the pulse rate of the user based on the ring finger in contact therewith. The GSR sensor 41 detects the GSR of the user based on the thumb or palm of the hand in contact with the one electrode 41a and the ring finger or little finger in contact with the other electrode 41b.

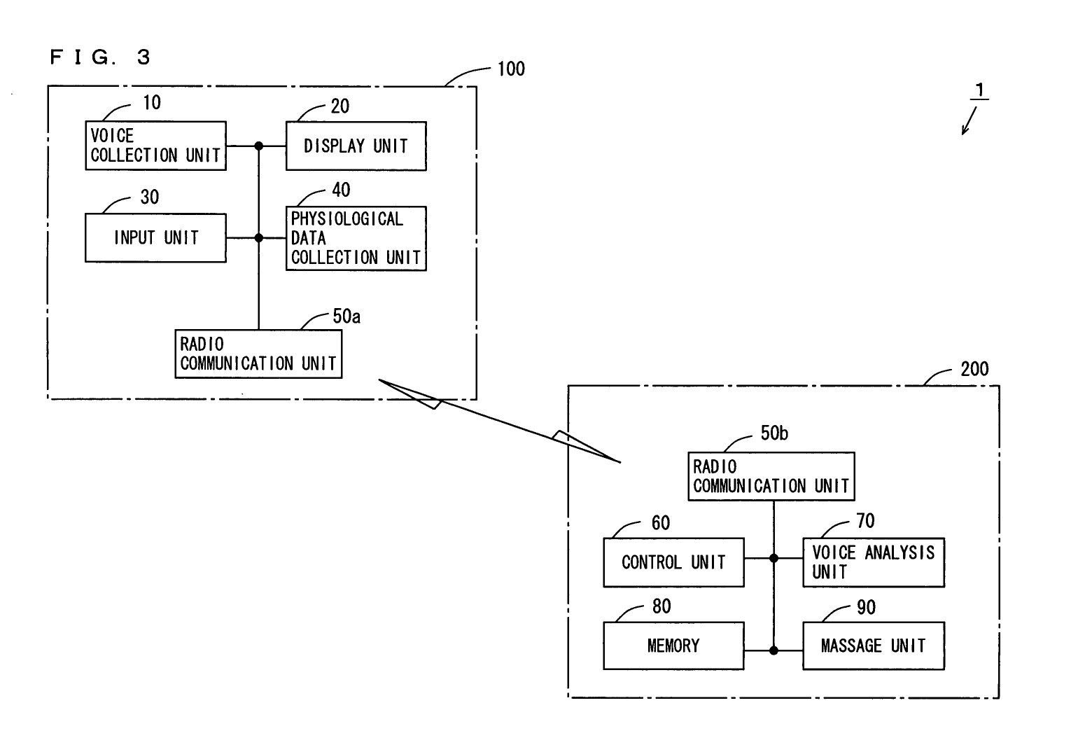

FIG. 3 is a block diagram showing the configuration of the relaxation system according to the embodiment of the present invention.

As mentioned above, the relaxation system 1 according to the embodiment comprises the remote controller 100 and the main body 200. The remote controller 100 includes a voice collection unit 10, a display 20, an input unit 30, a physiological data collection unit 40, and a radio communication unit 50a. The voice collection unit 10 includes the voice collection microphone 11 of FIG. 2, and the physiological data collection unit 40 includes the GSR sensor 41, pulse sensor 42, and skin temperature sensor 43 of FIG. 2.

The main body 200, on the other hand, includes a control unit 60, a voice analysis unit 70, a memory 80, a massage unit 90, and a radio communication unit 50b. The massage unit 90 includes the kneading balls 211 of FIG. 1.

With the remote controller 100, the user voice is input to the voice collection unit 10 via the voice collection microphone 11. The voice collection unit 10 generates a voice signal based on the input voice. The generated voice signal is transmitted by radio via the radio communication units 50a, 50b to the voice analysis unit 70.

The control unit 60 of the main body 200 makes the display 20 provide a question constituted by a given character string and images. An example of the display form on the display 20 will later be described.

Upon pressing of a button by the user, the input unit 30 transmits an answer to the above question, i.e., the Q&A data, via the radio communication units 50a, 50b to the control unit 60 of the main body 200.

As for the physiological data collection unit 40, it transmits the physiological data representing the skin temperature of the index finger detected by the skin temperature sensor 43, the pulse rate of the user detected by the pulse sensor 42, and the GSR of the user detected by the GSR sensor 41 as described above to the control unit 60 of the main body 200 via the radio communication units 50a, 50b.

The voice analysis unit 70 in the main body 200 performs voice analysis of the voice signal transmitted from the voice collection unit 10. As a result, the voice analysis unit 70 can obtain various analytical results, for example, on the tone, inflection, and jitter of the spoken voice, i.e., the voice data. The voice analysis unit 70 outputs this voice data to the control unit 60.

With each of the physiological data input via the radio communication units 50a, 50b from the physiological data collection unit 40 of the remote controller 100 and the voice data input from the voice analysis unit 70, the control unit 60 estimates physical and mental conditions of the user based on a physical condition table and a mental condition table to be described later.

With the estimated physical and mental conditions of the user, the control unit 60 then selects a relaxation technique level that is most suited to the current physical and mental conditions of the user based on a relaxation technique table.

The control unit 60 subsequently modifies the selected relaxation technique level based on the Q&A data input via the radio communication units 50a, 50b from the input unit 30 of the remote controller 100. Such modification to the level of the relaxation technique allows the user to relieve the stress effectively, according to his own physical and mental conditions. A specific example of the modification to the level of relaxation technique will later be illustrated.

After that, the control unit 60 controls the operation of the massage unit 90 based on the ultimately selected relaxation technique level. The massage unit 90 performs various kinds of relaxation operations for the user in response to a drive signal fed from the control unit 60. The relaxation operations may include, for example, stimulating the back or shoulders of the human body with the kneading balls 211 of FIG. 1.

Where the foregoing voice signal, physiological data, and Q&A data received by the radio communication unit 50b are analog data, an analog-digital (A/D) converter is incorporated into the configuration of the main body 200.

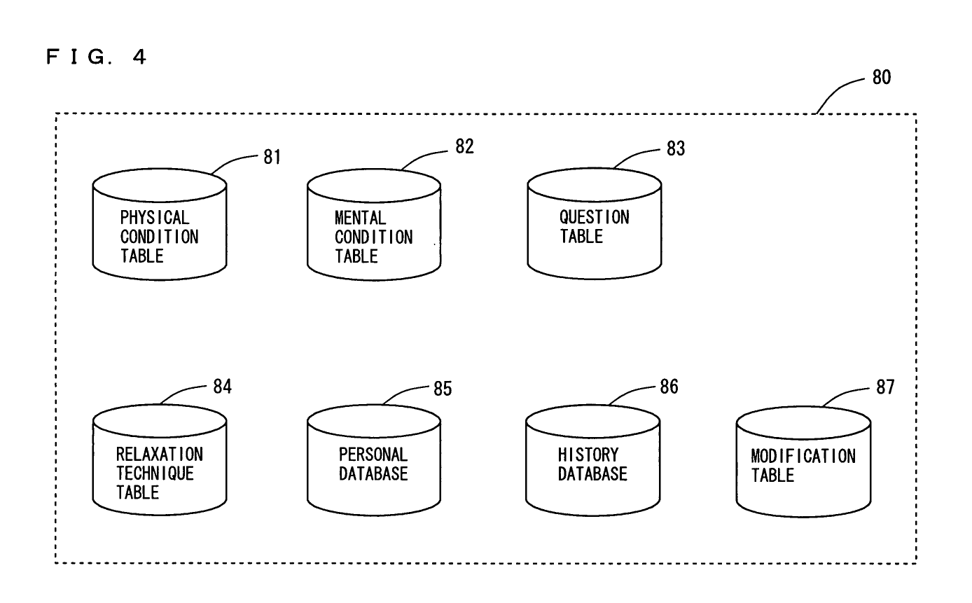

FIG. 4 is a diagram showing an example of the variety of tables and databases stored in the memory 80 of FIG. 3. The memory 80 stores a physical condition table 81, a mental condition table 82, a question table 83, a relaxation technique table 84, a personal database 85, a history database 86, and a modification table 87.

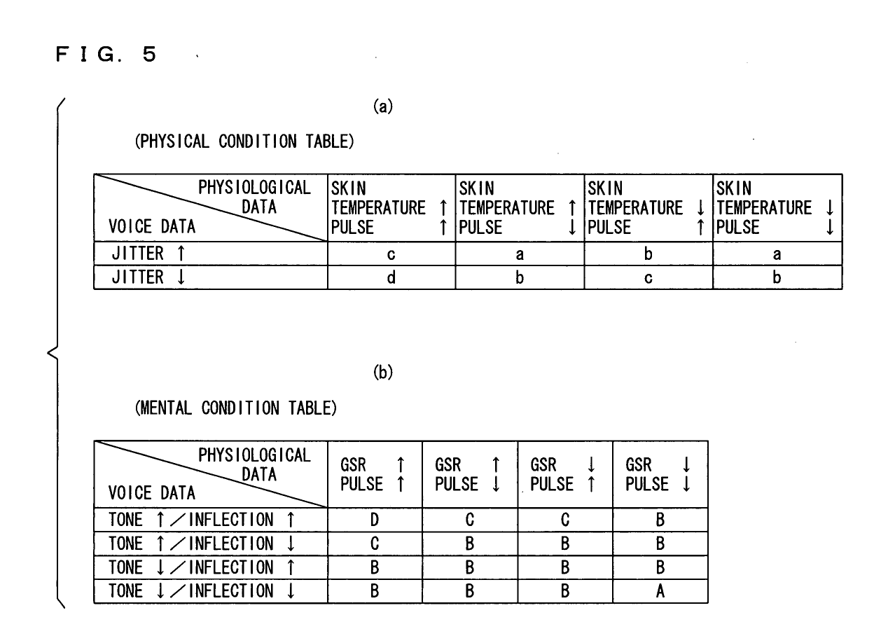

Each of the above tables and databases will be described in detail with reference to FIGS. 5 to 7. FIG. 5(a) shows an example of the physical condition table 81 of FIG. 4, and FIG. 5(b) shows an example of the mental condition table 82 of FIG. 4.

In the following description, the memory 80 stores a given reference value for each of the tone, inflection, and jitter of the spoken voice represented by the voice data, in addition to the variety of tables and databases shown in FIG. 4. The memory 80 also stores a given reference value for each of the skin temperature, pulse rate, and GSR represented by the physiological data. Moreover, the memory 80 stores a relaxation program to be described later although not shown in FIG. 4.

The control unit 60 determines whether each of the tone, inflection, and jitter of the spoken voice is greater or smaller than their given reference value, and also determines whether each of the skin temperature, pulse rate, and GSR of the physiological data is greater or smaller than their given reference value.

In FIG. 5, the arrow ↑ shows that each of the tone, inflection, and jitter of the spoken voice along with each of the skin temperature, pulse rate, and GSR are greater than their given reference values, whereas the arrow ↓ shows that each of the tone, inflection, and jitter of the spoken voice along with each of the skin temperature, pulse rate, and GSR are smaller than their given reference values.

As shown in the physical condition table 81 of FIG. 5(a), the physical condition of the user is estimated, for example, by being assigned to any of the four levels of “a”, “b”, “c”, and “d”. In this case, the physical condition “a” indicates being fine, “b” indicates somewhat fine, “c” indicates somewhat tired, and “d” indicates tired.

The control unit 60 determines whether the jitter is greater or smaller than its given reference value, and determines whether each of the skin temperature and pulse rate is greater or smaller than their given reference value. Based on each of the determinations, the control unit 60 estimates to which of the levels “a”, “b”, “c” and “d” the physical condition of the user may belong.

For example, where the jitter is greater than its given reference value, and each of the skin temperature and pulse rate is greater than their given reference value, the control unit 60 estimates that the user is in the physical condition level of “c” based on the physical condition table 81.

As shown in the mental condition table 82 of FIG. 5(b), the mental condition of the user is estimated, for example, by being assigned to any of the four levels of “A”, “B”, “C”, and “D”. In this case, the physical condition “A” indicates being relaxed, “B” indicates somewhat relaxed, “C” indicates somewhat stressed, and “D” indicates stressed.

The control unit 60 determines whether each of the tone and inflection is greater or smaller than their given reference value, and determines whether each of the pulse rate and GSR is greater or smaller than their given reference value. Based on each of the determinations, the control unit 60 estimates to which of the levels “A”, “B”, “C” and “D” the mental condition of the user may belong.

For example, where each of the tone and inflection is greater than their given reference value, and each of the pulse rate and GSR is greater than their given reference value, the control unit 60 estimates that the user is in the mental condition level of “D” based on the mental condition table 82.

It is preferred that each of the reference values used for the tone, inflection, and jitter, as well as the skin temperature, pulse rate and GSR, are obtained in advance when the user is in good physical and mental conditions.

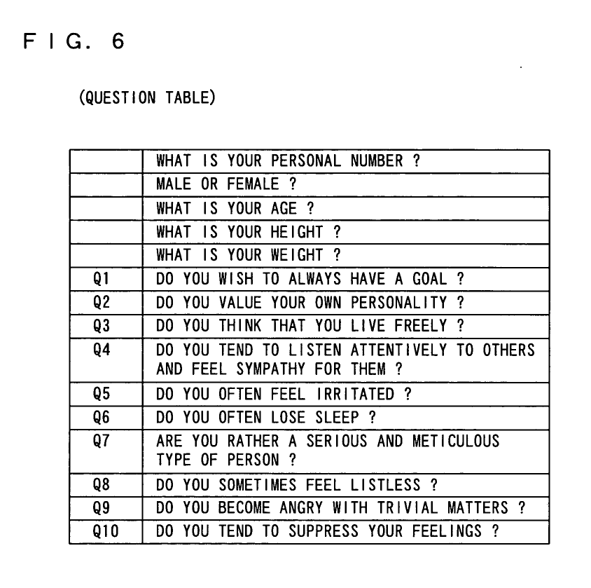

FIG. 6 shows an example of the question table 83 of FIG. 4. As shown in FIG. 4, the question table 83 stores in advance a plurality of questions. The control unit 60 thus makes the display 20 of the remote controller 100 provide a question based on the question table 83.

The user inputs an answer to each of the questions via the input unit 30 by manipulating the input unit 30. This results in the creation of Q&A data, i.e., answers to the questions as described above. Among the questions are, as shown in FIG. 6, the questions related to the identification of the user and the physical questions, such as “What is your personal number?”, “Male or Female?”, or “What is your height?”, along with the questions about the personality (mentality) of the user, such as “Do you often feel irritated?” or “Are you rather a serious and meticulous type of person?”.

With such inclusion of physical and mental questions, it is possible to modify the relaxation technique level described later that is selected based on the estimated physical and mental conditions to an optimum state.

In the embodiment, the questions in the question table 83 and the Q&A data are correlated with the relaxation technique levels to be modified (hereinafter referred to as modification levels) in a modification table 87 to be described later. The modification table 87 and modification levels will later be described.

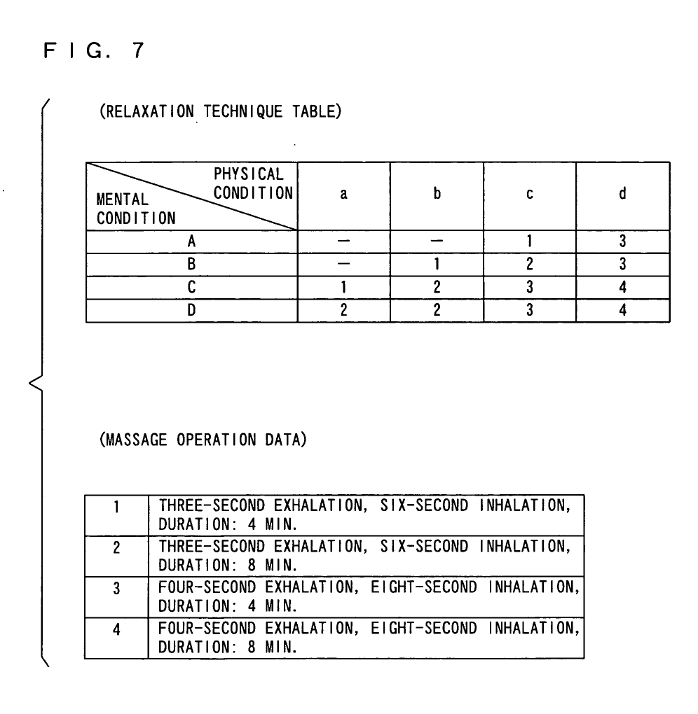

FIG. 7 shows an example of the relaxation technique table 84 of FIG. 4. As shown in FIG. 7, the relaxation techniques vary according to the four levels of “1”, “2”, “3”, and “4”, for example.

A stress relieving method for the user is set according to each of the respective four levels. Suppose, for example, that such stress relieving method for the user is to guide a respiration method by operating the kneading balls 211 of FIG. 1.

In this case, the user is made to exhale with the kneading balls 211 protruding from the backrest 210 toward the back of the user, and made to inhale with the kneading balls 211 drawn inside the backrest 210. As a result, by controlling the operation of the kneading balls 211, the user is guided into a given state of respiration.

Thus, when the control unit 60 has selected the relaxation technique level of “1” from the relaxation technique table 84 of FIG. 7, a respiration guidance is performed in which the user may exhale for three seconds and inhale for six seconds over four minutes.

When, on the other hand, the control unit 60 has selected the relaxation technique level of “4”, a respiration guidance is performed in which the user may exhale for four seconds and inhale for eight seconds over eight minutes. Data representing such operations of the massage unit 90 set according to the respective relaxation technique levels will be called massage operation data.

The foregoing modification table 87 and modification levels will now be described. In the modification table 87, there are three modification levels, for example, “+1”, “0”, and “−1”. The modification level “+1” here indicates that, when the control unit 60 has selected a specific relaxation technique level from the relaxation technique table 84, one is to be added to the selected level. In other words, when the control unit 60 has selected the relaxation technique level of “1”, what is actually performed for the user is the respiration guidance according to the relaxation technique level of “2”.

Similarly, the modification level “−1” indicates that, when the control unit 60 has selected a specific relaxation technique level from the relaxation technique table 84, one is to be subtracted from the selected level. The modification level “0” on the other hand indicates that, when the control unit 60 has selected a specific relaxation technique level from the relaxation technique table 84, no modification is to be made to the selected level.

The modification levels are correlated with the questions in the question table 83 and the Q&A data. For example, the modification level “+1” is correlated with a case in which many of negative questions among the question data are answered in the affirmative, whereas the modification level “−1” is correlated with a case in which many of the negative questions are answered in the negative. In addition, the modification level “0” is correlated with a case in which the answers to the negative questions are judged as standard.

A method of determining a modification level will specifically be described. In the following description, the memory 80 additionally stores a question and answer score table in which questions and their respective question and answer scores are set in advance.

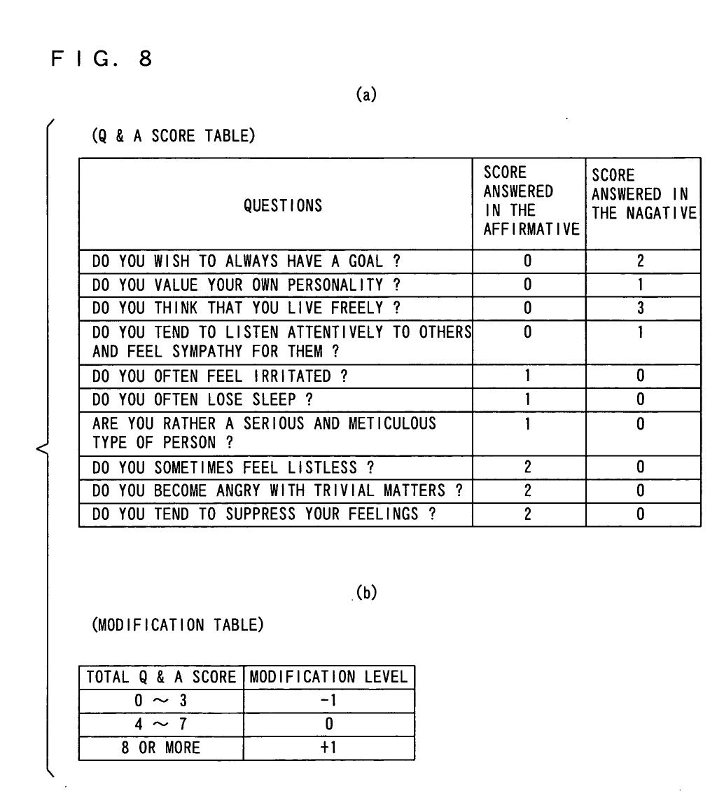

FIG. 8 is a table for use in illustrating a specific example of the method of determining a modification level. FIG. 8(a) shows the Q&A score table, and FIG. 8(b) shows the modification table.

As shown in FIG. 8(a), the Q&A score table contains preset questions and their respective Q&A scores. For example, when the user answers in the negative to the question: Do you always wish to have a goal?, then the score gains “two points”.

As a plurality of questions are presented to the user, the Q&A score of the user is added for each question. This results in the determination of a total value of the Q&A score of the user. Thus, the modification table of FIG. 8(b) determines a modification level based on the total value of the Q&A score.

According to the modification table of FIG. 8(b), the total Q&A score value of two points is correlated with the modification level of “−1”. Thus, when the control unit 60 has selected a specific relaxation technique level from the relaxation technique table 84, one is subtracted from the selected level.

The memory 80 may store a plurality of relaxation technique tables. When the memory 80 stores a plurality of relaxation technique tables, the control 60 may determine, by determining a modification level as described above, a relaxation technique table for use in selecting a relaxation technique level.

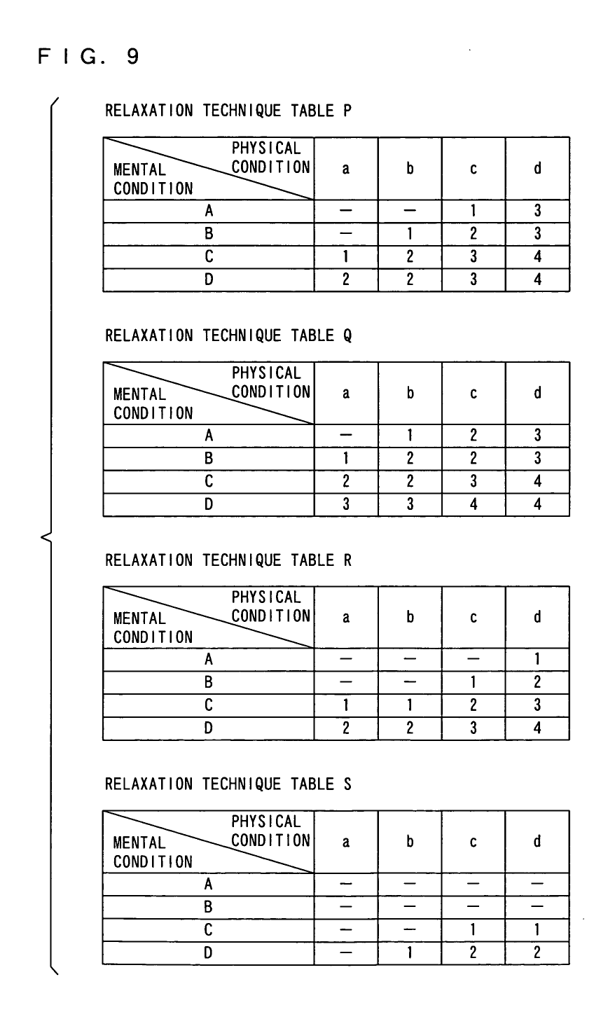

FIG. 9 shows examples of a plurality of relaxation technique tables stored in the memory 80 of FIG. 4. In each of the plurality of relaxation technique tables P to S of FIG. 9, the relaxation techniques vary according to the four levels of “1”, “2”, “3”, and “4”, as in FIG. 7.

In the respective relaxation technique tables P to S of FIG. 9, however, the relaxation technique levels correlated with their respective physical and mental conditions are set to be different from one table to another.

Suppose, for example, that the user is in the physical condition level of “c”, and also in the mental condition level of “C”.

In this case, according to the relaxation technique table P, the relaxation technique level is set to “3”. According to the relaxation technique table Q, the relaxation technique level is set to “3”, whereas according to the relaxation technique table R, the relaxation technique level is set to “2”. On the other hand, according to the relaxation technique table S, the relaxation technique level is set to “1”.

The modification table is used in order to determine one relaxation technique table among the plurality of relaxation technique tables P to S.

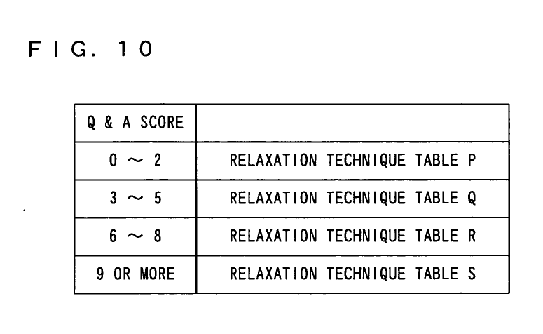

FIG. 10 shows an example of the modification table for determining one relaxation technique table among the plurality of relaxation technique tables P to S.

According to the modification table of FIG. 10, when the total value of the Q&A score is two points, the relaxation technique table P is selected. When, on the other hand, the total value of the Q&A score is four points, the relaxation technique table Q is selected.

Where the memory 80 stores a plurality of relaxation technique tables as described above, the relaxation technique table for use in selecting a relaxation technique level may be determined by the user manipulating the remote controller 100.

In this case, since the user can select a relaxation technique table, the user can relieve the stress of his body sufficiently, effectively, to his own taste.

The personal database 85 stores the histories of the above-mentioned voice data, physiological data, and Q&A data along with their reference values, based on the personal number represented by the Q&A data. The reference values of the respective voice data, physiological data, and Q&A data for each individual will be called basic data. The basic data is updated based on the histories of the voice data, physiological data, and Q&A data. A common average value of each of the voice data, physiological data, and Q&A data is for example set as an initial value of the basic data.

The history database 86 stores the personal number represented by the Q&A data, and also stores the relaxation technique levels the user has received as a history. The history of the stored relaxation technique levels will be called relaxation history data.

The control unit 60 is capable of reading the basic data for each person among a plurality of users or reading the relaxation history data with reference to the personal database 85 and the history database 86.

The relaxation program according to the embodiment will be described. Although the relaxation program according to the embodiment is stored in the memory 80, it may alternatively be stored in other recording medium, such as a hard disk, magnetic disk, or optical disk. In this case, the relaxation system 1 of FIG. 3 is provided with a driving device for driving a specific recording medium.

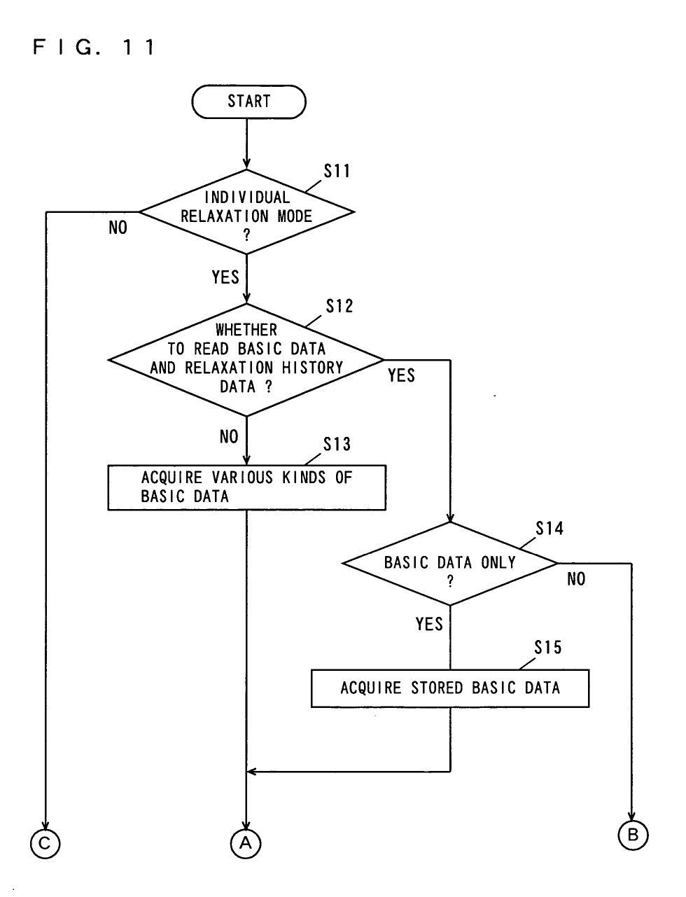

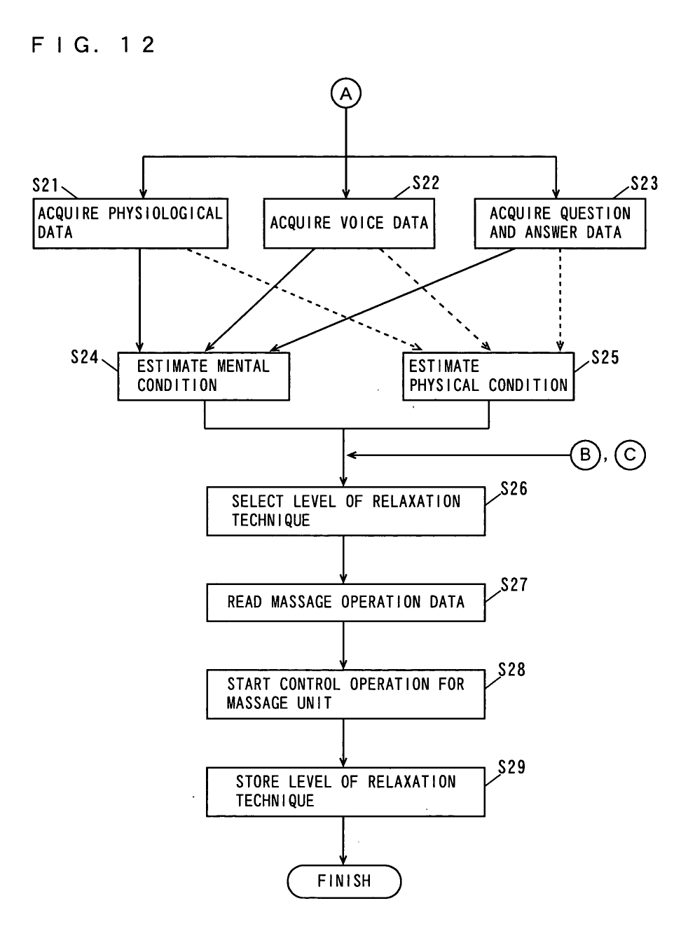

FIGS. 11 and 12 are flowcharts showing an example of the relaxation program according to the embodiment of the present invention.

Initially, the control unit 60 of FIG. 3 makes the user select via the remote controller 100 whether or not to receive a stress relieving method according to a relaxation technique suited to himself (an individual relaxation mode), followed by the determination of the answer (Step S11).

When the individual relaxation mode is selected, the control unit 60 makes the user select whether or not to read the basic data and relaxation history data related to himself, followed by the determination of the answer (Step S12).

Upon determination that the basic data and relaxation history data will not be read, the control unit 60 acquires the various kinds of basic data described above (Step S13). The acquisition of the basic data is performed similarly to the following operations of Steps S21 to S23.

After that, the control unit 60 acquires the current physiological data, voice data, and Q&A data of the user, as shown in Steps S21 to S23 of FIG. 12.

Upon determination that the user will read the basic data and the relaxation history data at Step S12, the control unit 60 makes the user select whether or not to read only the basic data of the basic data and relaxation history data, followed by the determination of the answer (Step S14). Upon determination that only the basic data will be read, the control unit 60 makes the user answer his own personal number to acquire the basic data stored in the personal database 85 of FIG. 4 (Step S15).

The acquisition of the physiological data shown in Step S21 of FIG. 12 is accomplished when the user grasps the remote controller 100 as described above, which results in the detection of the skin temperature, pulse rate, and GSR by each of the respective sensors.

The acquisition of the voice data shown in Step S22 of FIG. 12 is accomplished when the user reads a predetermined word or sentence provided on the display 20 of the remote controller 100 toward the voice collection microphone 11 of the voice collection unit 10.

The acquisition of the Q&A data shown in Step S23 of FIG. 12 is accomplished when the user manipulates the input unit 30 in response to a question provided on the display 20 of the remote controller 100 stored in the question table 83 of FIG. 4.

Although in the embodiment, the operations of Steps S21 to S23 above are performed in parallel, they may be separately performed in sequence.

The control unit 60 subsequently estimates a mental condition (Step S24) and physical condition (Step S25) based on the basic data acquired by the either operations of Step S13 or Step S15, and the current physiological data, voice data, and Q&A data of the user obtained by the operations of Steps S21 to S23. Although the operations of Steps S24, S25 are performed in parallel, they may be separately performed in sequence. The estimation of mental and physical conditions is done as in the manner described in FIG. 5.

Then, the control unit 60 selects a relaxation technique level based on the estimated physical and mental conditions (Step S26).

Even if the individual relaxation mode has not been selected at Step S11 above, the control unit 60 selects a relaxation technique level. The selection of a relaxation technique level in this case is done by making the user select a suitable relaxation technique level via the remote controller 100.

Even with the determination at Step S14 above that not only the basic data but also the relaxation history data would be read, the control unit 60 selects a relaxation technique level. The selection of a relaxation technique level in this case is done by the control unit 60, making the user answer his personal number via the remote controller 100 to acquire the basic data and the relaxation history data stored in the personal database 85 and the history database 86 of FIG. 4, thereby selecting a relaxation technique level among those the user has received in the past.

When a relaxation technique level has been selected, the control unit 60 reads massage operation data corresponding to the level (Step S27), to start the operation of controlling the massage unit 90 of FIG. 1 (Step S28). Upon completion of the control operation for the massage unit 90, the control unit 60 correlates the performed operation of controlling the massage unit 90, i.e., the relaxation technique level, with the personal number of the user, for storage into the history database 86 as relaxation history data (Step S29).

In the above flowcharts, the acquisition of the physiological data, voice data, and Q&A data is not performed during the operation of controlling the massage unit 90; however, each of the various data may be acquired by making the user constantly grasp the remote controller 100 during the operation of controlling the massage unit 90. In this case, stress relieving patterns for the user can be varied while the massage unit 90 are controlling the operation of the massage unit 90.

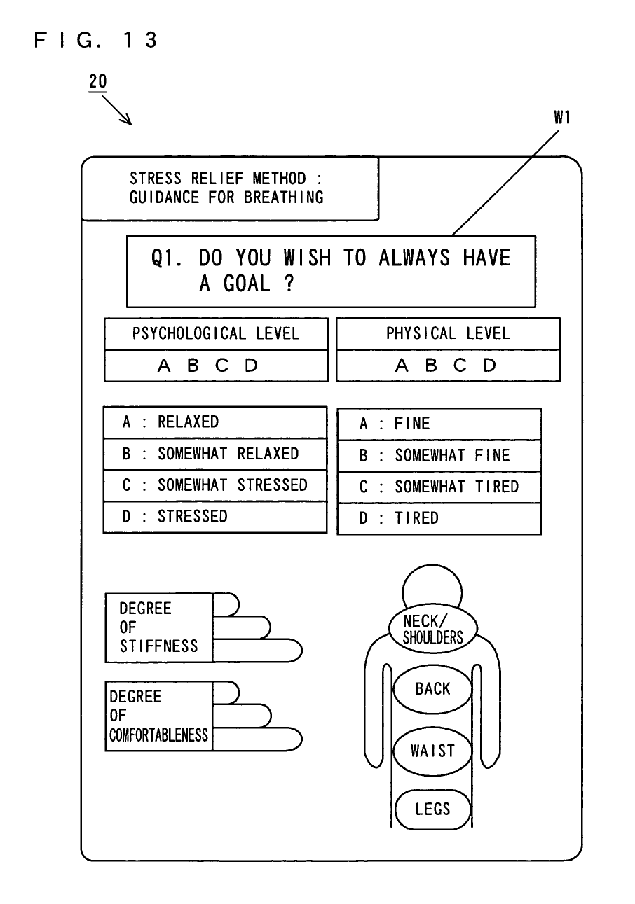

FIG. 13 is a schematic diagram showing an example of a display form on the display 20 of FIG. 2, during the questioning to the user.

As shown in FIG. 13, during the questioning, a question is provided on a character string display W1 of the display 20. The user answers in response to the question by pressing either of the Yes button 32 or No button 33 of FIG. 2.

In this manner, a question to the user is provided on the character string display W1 of the display 20, in response to which the user presses the Yes button 32 or No button 33 and the numeric buttons 34.

In the foregoing relaxation system according to the embodiment, the operation of the massage unit 90 of FIG. 1 is controlled based on the estimation results on physical and mental conditions; so that the stress of a human body can be sufficiently, effectively relieved according to his physical and mental conditions.

In addition, the control unit 60 of FIG. 3 estimates the physical condition of the user based on the voice data and physiological data obtained by the voice collection unit 10, input unit 30, and physiological data collection unit 40 of the remote controller 100 with reference to the physical condition table 81, and also estimates the mental condition of the user based on the voice data and physiological data with reference to the mental condition table 82. This facilitates the estimation of the physical and mental conditions of the user based on the various kinds of data obtained with the remote controller 100.

Furthermore, in the above embodiment, the physical and mental conditions of the user are estimated based on the voice data and physiological data; so that the estimation of the physical and mental conditions of the user is done in more detail and more accurately.

Furthermore, the modification level is determined according to the Q&A data, and a modification is made to the relaxation technique level selected from the relaxation technique table 84. This allows the user to relieve the stress of his body according to the physical and mental conditions of the user in more detail and more accurately.

Furthermore, the control unit 60 selects the most suitable level for the user among the plurality of relaxation technique levels based on his estimated physical and mental conditions, thereby controlling the massage unit 90 according to the selected relaxation technique level. Consequently, the user can relieve the stress of his body sufficiently, effectively, based on his physical and mental conditions.

Although in the embodiment, the Q&A data is used only for the selection of a modification level, it may also be used for the estimation of physical and mental conditions in FIG. 5. In this case, the estimation of the physical and mental conditions of the user is done in more detail and more accurately.

Finally, the massage unit 90 according to the embodiment of the present invention will be described in detail. As shown in FIG. 1, the massage unit 90 in the embodiment includes the plurality of kneading balls 211. With the operation of the plurality of kneading balls 211 controlled by the control unit 60, the timing for exhalation and inhalation of the user is guided into an optimum state so as to relieve the stress of his body.

In the above embodiment, the timing for exhalation and inhalation is set such that the proportion of the inhalation period to the exhalation period is “1:2”; however, there may be a stop period for exhalation and inhalation between the exhalation and inhalation periods.

Alternatively, a respiration cycle may be calculated based on the physiological data obtained by the physiological data collection unit 40 of FIG. 2, and the respiration cycle of the user may be controlled based on the calculated respiration cycle in such a manner as to gradually relieve the stress. Note that the respiration cycle should not necessarily be calculated as in the above, and it may alternatively be detected using a special sensor for detecting the respiration cycle.

The massage unit 90 may include a leg vibrator (e.g., a foot bath) instead of the kneading balls 211. In this case, the control unit 60 relieves the stress of a human body, for example by controlling the vibration given to the legs.

More specifically, the control unit 60 controls on/off of the leg vibrator, as will be shown below.

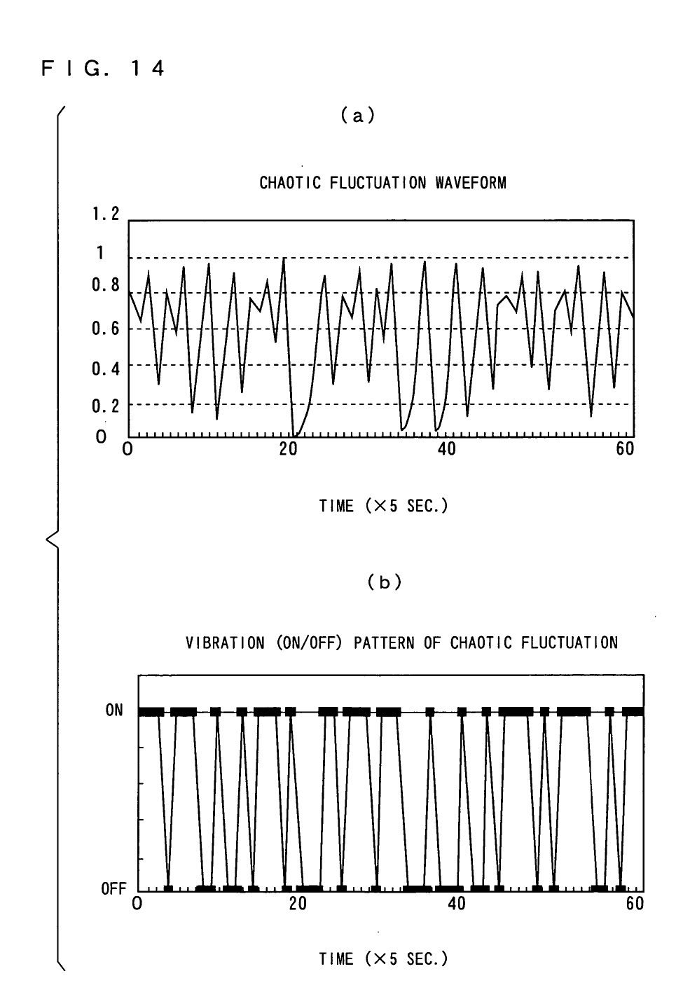

FIG. 14 are diagrams showing examples of chaotic fluctuation and on/off of the leg vibrator based on the chaotic fluctuation.

As shown in FIG. 14(a), the chaotic fluctuation is represented by a cycle of 1/fⁿ. In this example, the control unit 60 controls on/off of the leg vibrator as shown in FIG. 14(b).

The on/off of the leg vibrator is controlled by the control unit 60 based on a predetermined relaxation technique as in the above. In this case, the relaxation techniques vary according to a plurality of levels, for example, by varying the cycle of 1/fⁿ or varying the duration of vibration by the leg vibrator.

The operation of the leg vibrator is thus controlled based on the voice data, physiological data, and Q&A data obtained from the user, which allows the leg vibrator to relieve the stress of his body according to the current condition of the user. This allows the user to relieve the stress of his body sufficiently, effectively, as compared with a leg vibrator which constantly vibrates.

Note that, instead of on/off of the leg vibrator based on the chaotic fluctuation, the control unit 60 may add chaotic fluctuation to the vibration toward legs by the leg vibrator, and vary the chaotic fluctuation cycle of 1/fⁿ according to the estimated current physical and mental conditions of the user.

The massage unit 90 may include an illuminator instead of the kneading balls 211. In this case, the control unit 60 relieves the stress of his body, for example by controlling the condition of illumination provided by the illuminator in a room.

The illumination condition in a room is controlled by the control unit 60 based on a predetermined relaxation technique, as described above.

In this case, the relaxation techniques vary according to a plurality of levels, for example by varying the color temperature of the illumination. Note that the color temperature is a measure for indicating the tone of light, represented by K (kelvin).

Usually, light has a warm, reddish color with a low color temperature, whereas having a whitish color as the sunlight in the daytime with an increase in the color temperature. With a further increase in the color temperature, light changes to a bluish, fresh color.

Suppose that the relaxation techniques vary according to the four levels of“1”, “2”, “3”, and “4”, as described above. In this case, with the relaxation technique level being “4”, for example, the control unit 60 initially controls the condition of the illumination provided by the illuminator to a color temperature of approximately 3000 K, and gradually increases the color temperature to approximately 4000 K over a certain period of time.

With the relaxation technique level being “3”, the control unit 60 initially controls the condition of the illumination provided by the illuminator to a color temperature of approximately 3200 K, and gradually increases to approximately 4000 K over a certain period of time.

Further, with the relaxation technique level being “2”, the control unit 60 initially controls the condition of the illumination provided by the illuminator to a color temperature of approximately 3500 K, and gradually increases to approximately 4000 K over a certain period of time. With the relaxation technique level being “1”, the control unit 60 controls the illumination condition by the illuminator to a color temperature of approximately 4000 K from the beginning.

The illumination condition by the illuminator is thus controlled based on the voice data, physiological data, and Q&A data obtained from the user, so that the user can relieve the stress of his body, sufficiently, effectively.

Alternatively, the massage unit 90 may include a music provider instead of the kneading balls 211. In this case, the control unit 60 relieves the stress of a human body, for example by controlling the music produced by the music provider.

The music provided to the user is controlled by the control unit 60 based on a predetermined relaxation technique, as in the above case.

In this case, the relaxation techniques vary according to a plurality of. levels, for example, by selecting the kinds of music provided to the user.

Suppose that the relaxation techniques vary according to the four levels of “1”, “2”, “3”, and “4”, as described above. In this case, with the relaxation technique level being “4”, for example, the control unit 60 provides very calm music to the user by the music provider over a certain period of time.

With the relaxation technique level being “3”, the control unit 60 provides somewhat calm music to the user by the music provider over a certain period of time.

Further, with the relaxation technique level being “2”, the control unit 60 provides somewhat lively music to the user by the music provider over a certain period of time. With the relaxation technique level being “2”, the control unit 60 provides no music to the user.

The music provided to the user by the music provider is thus controlled based on the voice data, physiological data, and Q&A data obtained from the user, which allows the user to relieve the stress of his body, sufficiently, effectively.

In the above embodiment, the kneading balls 211 of the relaxation system 1 of FIG. 1 perform the respiration guidance to the user; however, by further controlling the operation of the kneading balls with the control unit 60, a massage according to the relaxed state of the user may be performed, according to a relaxation technique level.

The foregoing kneading balls 211, leg vibrator (e.g., a foot bath), illuminator, and music provider adaptable to the massage unit 90 may each be used in combination. In this case, the user can relieve the stress of his body sufficiently, effectively, based on his current physical and mental conditions, by means of the plurality of devices in the massage unit 90.

In the embodiment according to the present invention, the skin temperature of the user, pulse rate, GRS, voice, and user's answers to the questions correspond to information on the health condition of a user; the physical condition table 81 and mental condition table 82 in the memory 80 and the control unit 60 correspond to an estimator; the massage unit 90 including the kneading balls 211, leg vibrator, illuminator or music provider, and the control unit 60 correspond to a stress relieving apparatus; the physical condition levels “a”, “b”, “c”, and “d” as well as the mental condition levels “A”, “B”, “C”, and “D” correspond to estimation results; and the control unit 60 corresponds to a controller.

In addition, the physical condition of the user estimated by the physical condition table 81 based on the voice data and physiological data corresponds to a first correlation; the mental condition of the user estimated by the mental condition table 82 based on the voice data and physiological data corresponds to a second correlation; the relaxation technique selected by the relaxation technique table 84 based on the estimated physical and mental conditions of the user corresponds to a third correlation; the memory 80 corresponds to a correlation storage; the physiological data corresponds to information on the physiological data; and the voice data corresponds to information on the voice of a user. Moreover, the remote controller 100 corresponds to an acquisition device; the physiological data collection unit 40 of the remote controller 100, i.e., the GSR sensor 41, pulse sensor 42, and skin temperature sensor 43, along with the control unit 60, correspond to a physiological data acquisition device; and the voice collection unit 10 of the remote controller 100 corresponds to a voice acquisition device.

Furthermore, the display 20, input unit 30, and question table 83 correspond to a questioning device; the relaxation techniques according to various levels correspond to relaxation relieving techniques, the memory 80 or the relaxation technique table 84 within the memory 80 corresponds to a storage; the kneading balls 211 and the control unit 60 correspond to a respiration guidance device; the leg vibrator and the control unit 60 correspond to a chaotic fluctuation vibrator; the illuminator and the control unit 60 correspond to an illumination controller; the music provider and the controller 60 correspond to a music provider; the kneading balls 211 and the control unit 60 correspond to a massager; and the remote controller 100 correspond to an input device.

(Example of Other Configuration)

The relaxation system according to the present invention may alternatively have the following structure.

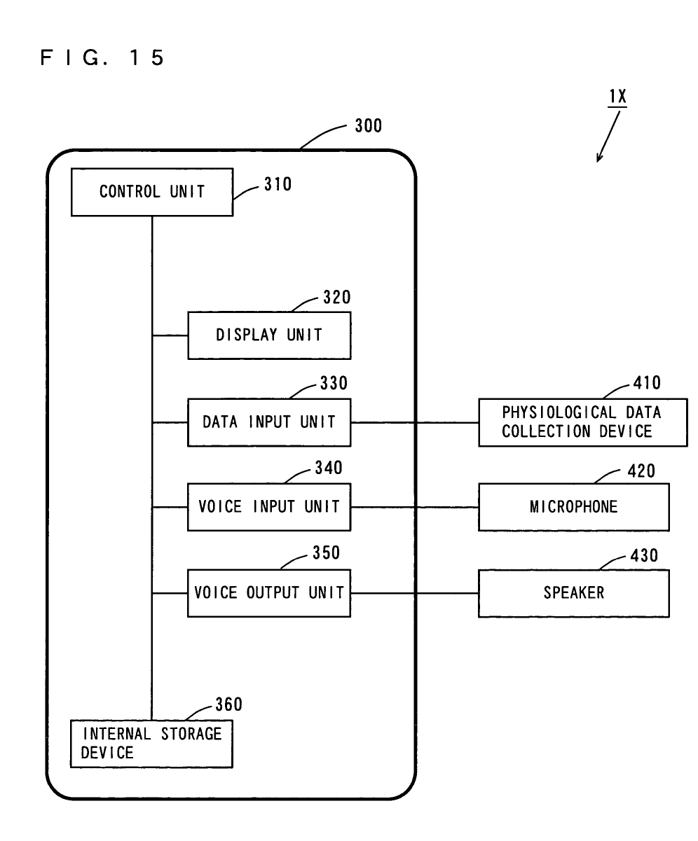

FIG. 15 is a block diagram showing an example of a relaxation system with a personal computer.

In this example, the relaxation system 1X includes a personal computer 300, a physiological data collection device 410, a microphone 420, and a speaker 430. The personal computer 300 comprises a control unit 310, a data input unit 330, a voice input unit 340, a voice output unit 350, and an internal storage device 360.

The data input unit 330 of the personal computer 300 is connected with the physiological data collection device 410, the voice input unit 340 connected with the microphone 420, and the voice output unit 350 connected with the speaker 430.

The control unit 310 of the personal computer 300 is, for example, composed of a CPU (Central Processing Unit) and a storage device or a microcomputer. In this example, the control unit 310 corresponds to the control unit 60 and the voice analysis unit 70 of FIG. 3.

The display 320 is composed of a cathode-ray tube display, liquid crystal display, organic electroluminescent display, plasma display or the like. In this example, the display 320 corresponds to the display 20 and the massage unit 90 of FIG. 3.

The internal storage device 360 is, for example, a hard disk drive. In this example, the internal storage device 360 corresponds to the memory 80 of FIG. 3. The internal storage device 360 thus stores the above-described relaxation program, variety of tables and databases shown in FIG. 4, and the like.

In this example of configuration, the relaxation program is stored in the internal storage device 360; note, however, that it may alternatively be stored in other recording medium, such as a hard disk, magnetic disk, or optical disk. In this case, the relaxation system 1X of FIG. 15 is provided with a driving device for driving a specific recording medium.

The physiological data collection device 410 connected to the data input unit 330 of the personal computer 300 corresponds to the physiological data collection unit 40 of FIG. 3. The physiological data collection device 410 thus includes, for example, the GSR sensor 41, pulse sensor 42, skin temperature sensor 43, and keyboard of FIG. 2.

The microphone 420 connected to the voice input unit 340 of the personal computer 300 corresponds to the microphone 11 of FIG. 2 and the voice collection unit 10 of FIG. 3. The speaker 430 connected to the voice output unit 350 of the personal computer 300 corresponds to the massage unit 90 of FIG. 3.

Each of the foregoing input units of the personal computer 300 corresponding to the physiological data collection device 410, microphone 420, and speaker 430, is composed of a serial port, parallel port, USB (Universal Serial Bus) port, or the like.

Note that the connections between the physiological data collection device 410, microphone 420, and speaker 430 and their respective input units in the personal computer 300 may be provided by radio.

The relaxation system of FIG. 15 operates for example as follows.

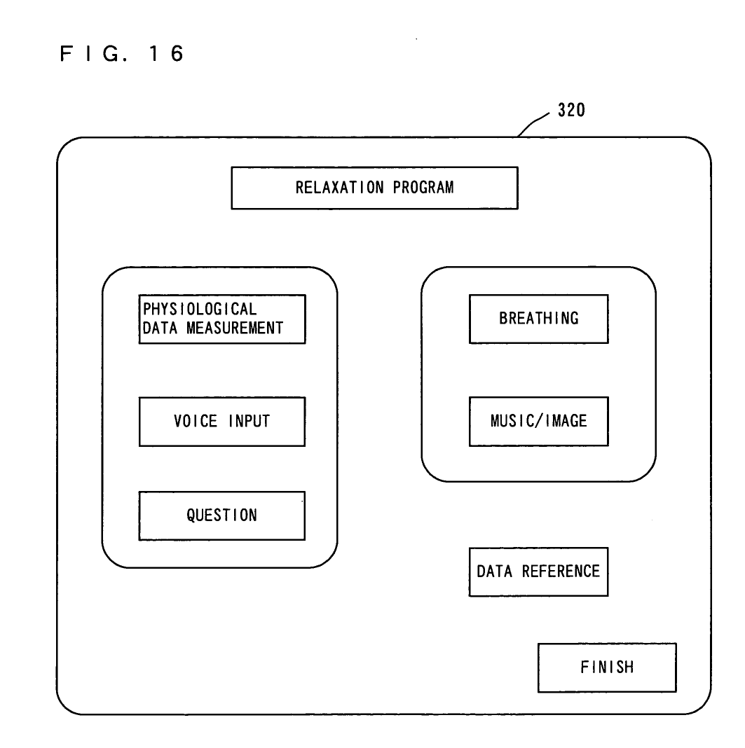

FIG. 16 is a diagram showing an example of a main screen provided on the display 320 of FIG. 15.

The main screen of FIG. 16 provides the display of a physiological data measurement button, voice input button, question button, respiration method button, music/image button, data reference button, and finish button.

Upon selection of the physiological data measurement button by the user, the physiological data collection device 410 transmits the physiological data detected by the GSR sensor 41, pulse sensor 42, and skin temperature sensor 43 of FIG. 2 to the control unit 310.

Upon selection of the voice input button by the user, the microphone 420 transmits a voice signal based on the voice of the user to the control unit 310. The control unit 310 performs voice analysis of the voice signal input from the microphone 420. This results in the creation of voice data.

The control unit 310 estimates the level of physical condition and the level of mental condition of the user based on the physiological data and the voice data, thereby selecting a relaxation technique level.

Upon selection of the question button by the user, the control unit 310 displays a question on the display 320 based on the question table 83 of FIG. 4 stored within the internal storage device 360. Then, the physiological data collection device 410 transmits an input signal obtained by the input operation through the keyboard to the control unit 310 as Q&A data. The control unit 310 determines the above-described modification level based on the Q&A data, to modify the relaxation technique level.

Upon selection of the respiration method button by the user, the control unit 310 performs a respiration guidance using the display 320 and speaker 430. Detailed operations of the display 320 and speaker 430 in this case will later be described.

Upon selection of the music/image button by the user, the control unit 310 provides through the display 320 and speaker 430 such image and music as the user may feel relaxed, according to the relaxation technique level.

Note that the music to be provided may be stored in advance within the internal storage device 360 according to the respective relaxation technique levels, as described above. In this case, the internal storage device 360 stores “very calm music”, “somewhat calm music”, “somewhat lively music” and so on, according to the respective relaxation technique levels.

The image and music may be concurrently provided to the user or may be separately provided to the user.

Still alternatively, the user may let the internal storage device 360 store in advance the image and music to be provided according to the respective relaxation technique levels. In this case, the user can select the image and music to his taste according to a selected relaxation technique level.

Upon selection of the data reference button by the user, the control unit 310 displays the estimated physical and mental condition levels, modification level, and the like on the display 320. Note that the voice data, physiological data, and Q&A data may appear on the display 320. The above-mentioned basic data may also appear on the display 320. Further, the histories of the voice data, physiological data, and Q&A data may appear thereon.

The selection of the finish button by the user completes the relaxation program.

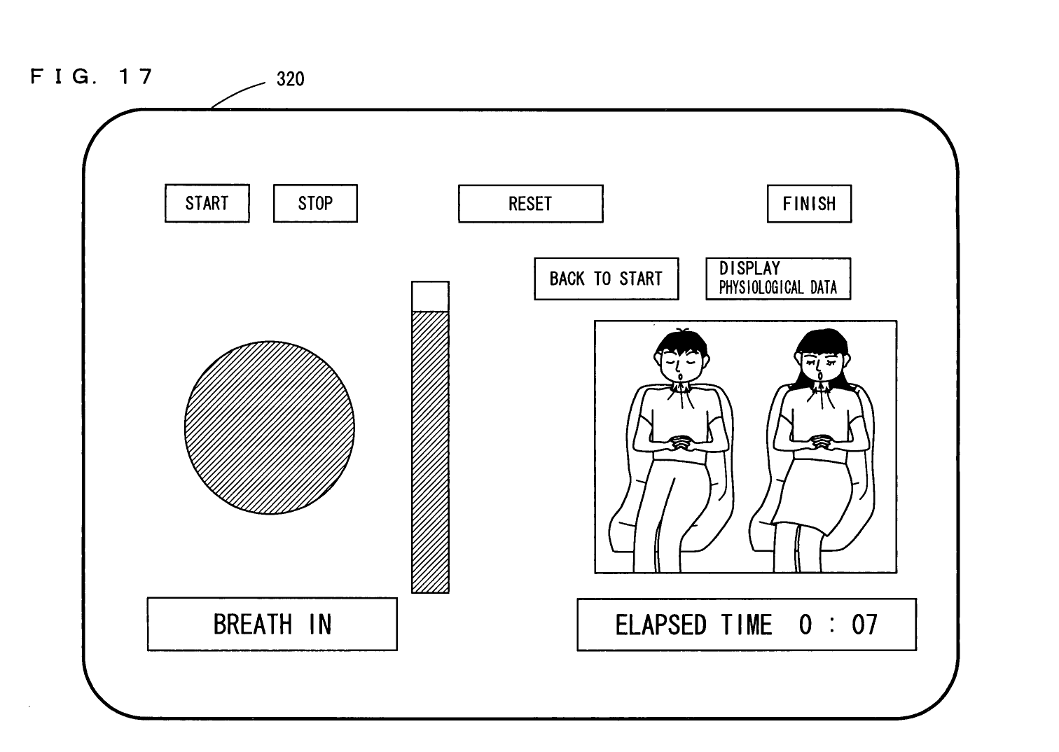

FIG. 17 is a diagram showing an example of a screen displayed when the relaxation system of FIG. 15 is performing the respiration guidance to the user. In FIG. 17, there is displayed a start button, stop button, reset button, back to start button, physiological data display button, and finish button, in addition to an image for instructing the user in respiration method.

By selecting the start button, the user can start the instruction in respiration method. By selecting the stop button, the user can stop the instruction in respiration method.

By selecting the reset button, the user can change his relaxation technique level. In this case, the relaxation technique levels, for example, appear on the display 320.

By selecting the back to start button, the user can restart the respiration guidance although still in progress.

By selecting the physiological data display button, the user is provided with the display of estimated physical condition level and mental condition level, a modification level, and the like on the display 320.

By selecting the finish button, the user completes the relaxation program.

On the display 320 of FIG. 17 is shown an animated instruction in respiration method to the user, in addition to the various buttons described above.

The shaded circle area of FIG. 17 increases with an instruction for the user to inhale, decreases with an instruction to exhale. The shaded block area in a bar graph form also increases with an instruction for the user to inhale, decreases with an instruction to exhale.

In FIG. 17, there is also shown an instruction in respiration method in letters, an elapsed time, and characters performing exhalation and inhalation.

Note that the instruction in respiration method to the user may be conducted through the speaker 430 by voice, instead of the display 320. In this case, voice is output from the speaker 430, such as “Breath in, breath in” or “Breath out, breath out.”

Although in the above example, the physiological data collection device 410 includes a keyboard, it may include a mouse instead. In this case, the GSR sensor 41, pulse sensor 42, and skin temperature sensor 43 of FIG. 2 may be attached to the mouse. In addition, the user's answers to the questions may be obtained by manipulating the mouse.

Further, in the above example, the user's answers to the questions are obtained by the input operation using the keyboard; however, the user's answers to the questions maybe input through the microphone 420 as a voice signal, which is subjected to voice analysis by the control unit 310 for the creation of Q&A data.

Although the present invention has been described and illustrated in detail, it is clearly understood that the same is by way of illustration and example only and is not to be taken by way of limitation, the spirit and scope of the present invention being limited only by the terms of the appended claims.

1. A relaxation system comprising:
an acquisition device that acquires information on the health condition of a user;
an estimator that estimates a physical condition and a mental condition based on the information acquired by said acquisition device;
a stress relieving apparatus that relives the stress of the user; and
a controller that controls said stress relieving apparatus based on estimation results by said estimator.

2. The relaxation system according to claim 1, further comprising a correlation storage that stores in advance a first correlation between the information acquired by said acquisition device and the physical condition and a second correlation between the information acquired by said acquisition device and the mental condition, wherein
said estimator extracts the physical and mental conditions based on said first and second correlations stored in said correlation storage.

3. The relaxation system according to claim 1, wherein said acquisition device includes a physiological data acquisition device that acquires information on the physiological data of the user, and
said information on the health condition includes the information acquired by said physiological data acquisition device.

4. The relaxation system according to claim 1, wherein
said acquisition device includes a voice acquisition device that acquires information on the voice of the user, and
said information on the health condition includes the information acquired by said voice acquisition device.

5. The relaxation system according to claim 1, wherein
said acquisition device includes a questioning device that questions the user while inputting an answer to the question, and
said information on the health condition includes the answer input by said questioning device.

6. The relaxation system according to claim 1, further comprising a storage that stores in advance a plurality of stress relieving techniques, wherein
said controller selects any of said plurality of stress relieving techniques stored in said storage based on the physical and mental conditions estimated by said estimator, to control said stress relieving apparatus according to the selected stress relieving technique.

7. The relaxation system according to claim 2, wherein
said correlation storage additionally stores a third correlation between physical and mental conditions and a plurality of stress relieving techniques for relieving the stress of the user.

8. The relaxation system according to claim 7, wherein
said controller selects any of said plurality of stress relieving techniques with reference to said third correlation stored in said correlation storage based on the physical and mental conditions estimated by said estimator, to control said stress relieving apparatus according to the selected stress relieving technique.

9. The relaxation system according to claim 7, wherein
said acquisition device includes a questioning device that questions the user while inputting an answer to the question,
said information on the health condition includes the answer input by said questioning device, and
said controller modifies said third correlation based on the answer input by said questioning device, to select any of said stress relieving techniques with reference to the modified third correlation based on the physical and mental conditions estimated by said estimator.

10. The relaxation system according to claim 2, wherein
said acquisition device includes a questioning device that questions the user while inputting an answer to the question,
said information on the health condition includes the answer input by said questioning device,
said correlation storage additionally stores a plurality of third correlations between the physical and mental conditions and a plurality of stress relieving techniques for relieving the stress of the user, and
said controller selects any of said plurality of third correlations based on the answer input by said questioning device, to select any of said plurality of stress relieving techniques with reference to the third correlation selected based on the physical and mental conditions estimated by said estimator, for controlling said stress relieving apparatus according to the selected stress relieving technique.

11. The relaxation system according to claim 10, further comprising an input device with which the user can select any of said plurality of third correlations, wherein
said controller controls said stress relieving apparatus according to the stress relieving technique based on the third correlation selected by the user manipulating said input device.

12. The relaxation system according to claim 1, wherein
said stress relieving apparatus includes at least one of a respiration guidance device that controls the respiration of the user, a chaotic fluctuation vibrator that gives vibration based on chaotic fluctuation to the user, an illumination controller that controls the condition of illumination, and a music provider that provides the user with music.

13. The relaxation system according to claim 1, wherein
said stress relieving apparatus is a massager.

14. A relaxation method comprising the steps of:
acquiring information on the health condition of a user;
estimating a physical condition and a mental condition based on said acquired information; and
relieving the stress of the user based on estimation results on said physical and mental conditions.

15. A computer-executable relaxation program which allows said computer to execute the processes of:
acquiring information on the health condition of a user;
estimating a physical condition and a mental condition based on said acquired information; and
relieving the stress of the user based on estimation results on said physical and mental conditions.
